(19) United States
(12) Patent Application Publication
(10) Pub. No.: US 2020/0246190 A1
(43) Pub. Date: Aug. 6, 2020

(54) DRAPE STRIP HAVING SELECTABLE ADHESIVE

(71) Applicant: KCI Licensing, Inc., San Antonio, TX (US)

(72) Inventors: James A. LUCKEMEYER, San Antonio, TX (US); Christopher Brian LOCKE, Bournemouth (GB)

(21) Appl. No.: 16/748,183

(22) Filed: Jan. 21, 2020

Related U.S. Application Data
(60) Provisional application No. 62/800,043, filed on Feb. 1, 2019.

(51) Int. Cl. A61F 13/00; A61B 46/20; A61M 1/00; A61F 13/02

(52) U.S. Cl. CPC A61F 13/00068; A61B 46/20; A61M 1/0088; A61F 13/0253; A61F 2013/00587; A61F 13/0269; A61B 2046/205; A61F 2013/00863; A61F 13/0259

(57) ABSTRACT

A dressing for treating a tissue site with negative pressure, a sealing tape, methods of use and methods of manufacturing the sealing tape are described. The dressing includes a tissue interface configured to be positioned adjacent to the tissue site and a sealing member configured to be positioned over the tissue interface and the tissue site to form a sealed environment. The dressing also includes a sealing tape having a carrier layer having a first side and a second side, a silicone adhesive layer bonded to the first side of the carrier layer, and an acrylic adhesive layer bonded to the second side of the carrier layer. A plurality of holes extend through the silicone adhesive layer, the acrylic adhesive layer, and the carrier layer. A first release liner is coupled to silicone adhesive layer, and a second release liner is coupled to the acrylic adhesive layer.

### RELATED APPLICATIONS

This application claims the benefit, under 35 U.S.C. § 119(e), of the filing of U.S. Provisional Patent Application Ser. No. 62/800,043, entitled “DRAPE STRIP HAVING SELECTABLE ADHESIVE,” filed Feb. 1, 2019, which is incorporated herein by reference for all purposes.

### TECHNICAL FIELD

The invention set forth in the appended claims relates generally to tissue treatment systems and more particularly, but without limitation, to a drape strip having selectable adhesives.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for sealing a dressing in a negative-pressure therapy environment are set forth in the appended claims. Illustrative embodiments are also provided to enable a person skilled in the art to make and use the claimed subject matter.

For example, in some embodiments, a sealing tape having an acrylic adhesive on a first side and a silicone adhesive on a second side. A plurality of holes are formed in the sealing tape through the acrylic adhesive and the silicone adhesive. A release liner can be coupled to the acrylic adhesive, and a release liner can be coupled to the silicone adhesive. The release liners can include handling bars.

More generally, a dressing for treating a tissue site with negative pressure is described. The dressing can include a tissue interface configured to be positioned adjacent to the tissue site and a sealing member configured to be positioned over the tissue interface and the tissue site to form a sealed environment. The dressing can also include a sealing tape having a carrier layer having a first side and a second side, a silicone adhesive layer bonded to the first side of the carrier layer, and an acrylic adhesive layer bonded to the second side of the carrier layer. A plurality of holes can extend through the silicone adhesive layer, the acrylic adhesive layer, and the carrier layer. A first release liner can be coupled to silicone adhesive layer, and a second release liner can be coupled to the acrylic adhesive layer.

Alternatively, other example embodiments may describe a drape strip. The drape strip can include a substrate having a first side and a second side, a sealing layer bonded to the first side of the substrate, and a bonding layer bonded to the second side of the substrate. A plurality of apertures can extend through the sealing layer, the bonding layer, and the substrate. A first protective layer can be coupled to sealing layer, and a second protective layer can be coupled to the bonding layer.

A method of manufacturing a sealing tape is described. A carrier layer having a first side and a second side is provided. A silicone adhesive layer can be bonded to the first side of the carrier layer, and an acrylic adhesive layer can be bonded to the second side of the carrier layer. A plurality of holes can be formed extending through the silicone adhesive layer, the acrylic adhesive layer, and the carrier layer. A first release liner can be releasably to silicone adhesive layer, and a second release liner can be releasably coupled to the acrylic adhesive layer.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is a sectional view with a portion shown in elevation of a negative-pressure system that may be associated with some embodiments of the therapy system of FIG. 1;

FIG. 3 is a plan view of a dressing illustrating additional details that may be associated with some embodiments of the negative-pressure therapy system of FIG. 2;

FIG. 4 is a perspective exploded view illustrating details of a drape strip that may be associated with some embodiments of the dressing of FIG. 3;

FIG. 5 is a perspective view illustrating additional details that may be associated with some embodiments of the drape strip of FIG. 4;

FIG. 6 is a plan view illustrating additional details of a portion of another drape strip that may be associated with some embodiments of the dressing of FIG. 3;

FIG. 7 is a plan view illustrating additional details of a portion of another drape strip that may be associated with some embodiments of the dressing of FIG. 3;

FIG. 8 is a plan view illustrating additional details of a portion of another drape strip that may be associated with some embodiments of the dressing of FIG. 3; and

FIG. 9 is a plan view illustrating additional details of a portion of another drape strip that may be associated with some embodiments of the dressing of FIG. 3.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

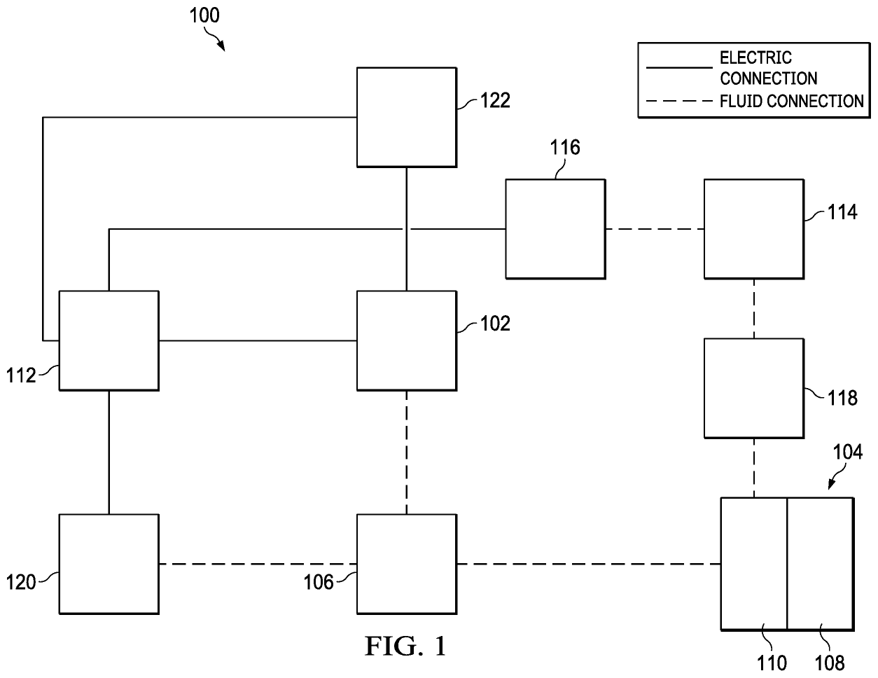

FIG. 1 is a simplified functional block diagram of an example embodiment of a therapy system 100 that can provide negative-pressure therapy with instillation of topical treatment solutions to a tissue site in accordance with this specification. The therapy system 100 may include a source or supply of negative pressure, such as a negative-pressure source 102, and one or more distribution components. A distribution component is preferably detachable and may be disposable, reusable, or recyclable. A dressing, such as a dressing 104, and a fluid container, such as a container 106, are examples of distribution components that may be associated with some examples of the therapy system 100. As illustrated in the example of FIG. 1, the dressing 104 may comprise or consist essentially of a tissue interface 108, a cover 110, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104. For example, such a dressing interface may be a SENSAT.R.A.C.™ Pad available from Kinetic Concepts, Inc. of San Antonio, Tex.

The therapy system 100 may also include a regulator or controller, such as a controller 112. Additionally, the therapy system 100 may include sensors to measure operating parameters and provide feedback signals to the controller 112 indicative of the operating parameters. As illustrated in FIG. 1, for example, the therapy system 100 may include a first sensor 120 and a second sensor 122 coupled to the controller 112.

The therapy system 100 may also include a source of instillation solution. For example, a solution source 114 may be fluidly coupled to the dressing 104, as illustrated in the example embodiment of FIG. 1. The solution source 114 may be fluidly coupled to a positive-pressure source such as a positive-pressure source 116, a negative-pressure source such as the negative-pressure source 102, or both in some embodiments. A regulator, such as an instillation regulator 118, may also be fluidly coupled to the solution source 114 and the dressing 104 to ensure proper dosage of instillation solution (e.g. saline) to a tissue site. For example, the instillation regulator 118 may comprise a piston that can be pneumatically actuated by the negative-pressure source 102 to draw instillation solution from the solution source during a negative-pressure interval and to instill the solution to a dressing during a venting interval. Additionally or alternatively, the controller 112 may be coupled to the negative-pressure source 102, the positive-pressure source 116, or both, to control dosage of instillation solution to a tissue site. In some embodiments, the instillation regulator 118 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the controller 112, the solution source 114, and other components into a therapy unit.

In general, components of the therapy system 100 may be coupled directly or indirectly. For example, the negative-pressure source 102 may be directly coupled to the container 106 and may be indirectly coupled to the dressing 104 through the container 106. Coupling may include fluid, mechanical, thermal, electrical, or chemical coupling (such as a chemical bond), or some combination of coupling in some contexts. For example, the negative-pressure source 102 may be electrically coupled to the controller 112 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 102, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 102 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 112, may be a microprocessor or computer programmed to operate one or more components of the therapy system 100, such as the negative-pressure source 102. In some embodiments, for example, the controller 112 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 108, for example. The controller 112 is also preferably configured to receive one or more input signals, such as a feedback signal, and programmed to modify one or more operating parameters based on the input signals.

Sensors, such as the first sensor 120 and the second sensor 122, are generally known in the art as any apparatus operable to detect or measure a physical phenomenon or property, and generally provide a signal indicative of the phenomenon or property that is detected or measured. For example, the first sensor 120 and the second sensor 122 may be configured to measure one or more operating parameters of the therapy system 100. In some embodiments, the first sensor 120 may be a transducer configured to measure pressure in a pneumatic pathway and convert the measurement to a signal indicative of the pressure measured. In some embodiments, for example, the first sensor 120 may be a piezo-resistive strain gauge. The second sensor 122 may optionally measure operating parameters of the negative-pressure source 102, such as a voltage or current. Preferably, the signals from the first sensor 120 and the second sensor 122 are suitable as an input signal to the controller 112, but some signal conditioning may be appropriate in some embodiments. For example, the signal may need to be filtered or amplified before it can be processed by the controller 112. Typically, the signal is an electrical signal, but may be represented in other forms, such as an optical signal.

The tissue interface 108 can be generally adapted to partially or fully contact a tissue site. The tissue interface 108 may take many forms, and may have many sizes, shapes, or thicknesses, depending on a variety of factors, such as the type of treatment being implemented or the nature and size of a tissue site. For example, the size and shape of the tissue interface 108 may be adapted to the contours of deep and irregular shaped tissue sites. Any or all of the surfaces of the tissue interface 108 may have an uneven, coarse, or jagged profile.

In some embodiments, the tissue interface 108 may comprise or consist essentially of a manifold. A manifold in this context may comprise or consist essentially of a means for collecting or distributing fluid across the tissue interface 108 under pressure. For example, a manifold may be adapted to receive negative pressure from a source and distribute negative pressure through multiple apertures across the tissue interface 108, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 108 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 108 may also vary according to needs of a prescribed therapy. For example, the tensile strength of foam may be increased for instillation of topical treatment solutions. The 25% compression load deflection of the tissue interface 108 may be at least 0.35 pounds per square inch, and the 65% compression load deflection may be at least 0.43 pounds per square inch. In some embodiments, the tensile strength of the tissue interface 108 may be at least 10 pounds per square inch. The tissue interface 108 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 108 may be reticulated polyurethane foam such as found in GRANUFOAM™ dressing or V.A.C. VERAFLO™ dressing, both available from Kinetic Concepts, Inc. of San Antonio, Tex.

The thickness of the tissue interface 108 may also vary according to needs of a prescribed therapy. For example, the thickness of the tissue interface may be decreased to reduce tension on peripheral tissue. The thickness of the tissue interface 108 can also affect the conformability of the tissue interface 108. In some embodiments, a thickness in a range of about 5 millimeters to 10 millimeters may be suitable.

The tissue interface 108 may be either hydrophobic or hydrophilic. In an example in which the tissue interface 108 may be hydrophilic, the tissue interface 108 may also wick fluid away from a tissue site, while continuing to distribute negative pressure to the tissue site. The wicking properties of the tissue interface 108 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Tex. Other hydrophilic foams may include those made from polyether. Other foams that may exhibit hydrophilic characteristics include hydrophobic foams that have been treated or coated to provide hydrophilicity.

In some embodiments, the tissue interface 108 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 108 may further serve as a scaffold for new cell-growth, or a scaffold material may be used in conjunction with the tissue interface 108 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 110 may provide a bacterial barrier and protection from physical trauma. The cover 110 may also be constructed from a material that can reduce evaporative losses and provide a fluid seal between two components or two environments, such as between a therapeutic environment and a local external environment. The cover 110 may comprise or consist of, for example, an elastomeric film or membrane that can provide a seal adequate to maintain a negative pressure at a tissue site for a given negative-pressure source. The cover 110 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 110 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 110 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minn.; polyurethane (PU) drape, commercially available from Avery Dennison Corporation, Pasadena, Calif.; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 110 may comprise INSPIRE 2301 having an MVTR (upright cup technique) of 2600 g/m²/24 hours and a thickness of about 30 microns.

An attachment device may be used to attach the cover 110 to an attachment surface, such as undamaged epidermis, a gasket, or another cover. The attachment device may take many forms. For example, an attachment device may be a medically-acceptable, pressure-sensitive adhesive configured to bond the cover 110 to epidermis around a tissue site. In some embodiments, for example, some or all of the cover 110 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The solution source 114 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

In operation, the tissue interface 108 may be placed within, over, on, or otherwise proximate to a tissue site. If the tissue site is a wound, for example, the tissue interface 108 may partially or completely fill the wound, or it may be placed over the wound. The cover 110 may be placed over the tissue interface 108 and sealed to an attachment surface near a tissue site. For example, the cover 110 may be sealed to undamaged epidermis peripheral to a tissue site. Thus, the dressing 104 can provide a sealed therapeutic environment proximate to a tissue site, substantially isolated from the external environment, and the negative-pressure source 102 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 108 in the sealed therapeutic environment can induce macro-strain and micro-strain in the tissue site. Negative pressure can also remove exudate and other fluid from a tissue site, which can be collected in container 106.

In some embodiments, the controller 112 may receive and process data from one or more sensors, such as the first sensor 120. The controller 112 may also control the operation of one or more components of the therapy system 100 to manage the pressure delivered to the tissue interface 108. In some embodiments, controller 112 may include an input for receiving a desired target pressure and may be programmed for processing data relating to the setting and inputting of the target pressure to be applied to the tissue interface 108. In some example embodiments, the target pressure may be a fixed pressure value set by an operator as the target negative pressure desired for therapy at a tissue site and then provided as input to the controller 112. The target pressure may vary from tissue site to tissue site based on the type of tissue forming a tissue site, the type of injury or wound (if any), the medical condition of the patient, and the preference of the attending physician. After selecting a desired target pressure, the controller 112 can operate the negative-pressure source 102 in one or more control modes based on the target pressure and may receive feedback from one or more sensors to maintain the target pressure at the tissue interface 108.

In some embodiments, the controller 112 may have a continuous pressure mode, in which the negative-pressure source 102 is operated to provide a constant target negative pressure for the duration of treatment or until manually deactivated. Additionally or alternatively, the controller may have an intermittent pressure mode. For example, the controller 112 can operate the negative-pressure source 102 to cycle between a target pressure and atmospheric pressure. For example, the target pressure may be set at a value of 135 mmHg for a specified period of time (e.g., 5 min), followed by a specified period of time (e.g., 2 min) of deactivation. The cycle can be repeated by activating the negative-pressure source 102, which can form a square wave pattern between the target pressure and atmospheric pressure. In some embodiments, the controller 112 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 112, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 112 may receive and process data, such as data related to instillation solution provided to the tissue interface 108. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The controller 112 may also control the fluid dynamics of instillation by providing a continuous flow of solution or an intermittent flow of solution.

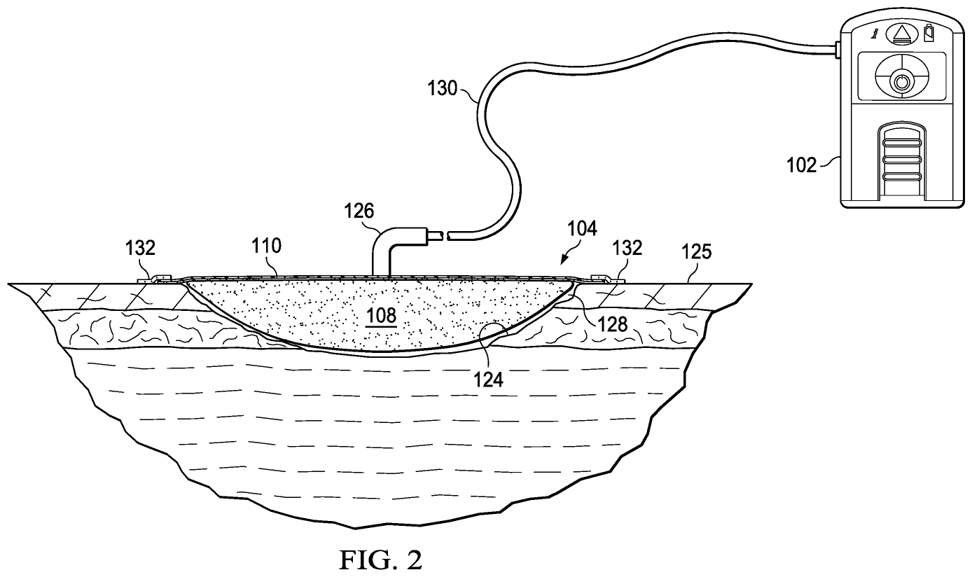

FIG. 2 is a sectional view with a portion shown in elevation of an example embodiment of the therapy system 100 for providing negative-pressure therapy. The tissue interface 108 may be disposed adjacent to a tissue site 124. The cover 110 can be disposed over the tissue interface 108 and the tissue site 124 and sealed to tissue surrounding the tissue site 124 to form a sealed therapeutic environment 128. In some embodiments, a connector 126 may be used to fluidly couple a fluid conductor, such as a tube 130 to the sealed therapeutic environment 128. The tube 130 can also be coupled to the negative-pressure source 102. The negative pressure developed by the negative-pressure source 102 may be delivered through the tube 130 to the connector 126. In one illustrative embodiment, the connector 126 may be a T.R.A.C.® Pad or Sensa T.R.A.C.® Pad available from KCI of San Antonio, Tex. The connector 126 allows the negative pressure to be delivered to the sealed therapeutic environment 128. In other exemplary embodiments, the connector 126 may also be a tube inserted through the cover 110. The negative pressure may also be generated by a device directly coupled to the cover 110, such as a micropump.

The provision of negative-pressure therapy with therapy systems, such as the therapy system 100, is increasingly being performed with smaller therapy devices that use battery power rather than a connection to an electrical outlet. Use of battery power decreases the total power supply available to a therapy device. As a result, power drains that would be considered negligible in a device powered through an electrical outlet connection may significantly reduce the ability of the therapy device to provide therapy. A power drain refers to operation of the therapy device that requires use of electrical power, for example, operation of a pump to generate negative pressure. Power drains may be caused by low-level dressing leaks, for example. A low-level dressing leak can drain power from a battery of a therapy device by repeatedly triggering operation of the therapy device to maintain the necessary negative pressure at the tissue site. Power drains can shorten the useful life of the therapy device by draining the device battery faster, requiring more frequent disposal of the device, recharging of the battery, or battery replacement. Leak detection techniques may help to identify some leaks that may be sealed by the user; however, low-level leaks will challenge the most sensitive leak detection systems and may often go undetected.

Low-level dressing leaks may occur between a drape and epidermis surrounding a tissue site if the drape fails to completely seal to the epidermis. Some negative-pressure dressings can leak after initial placement of the dressing, most often occurring in anatomical locations having many contours. Negative-pressure dressings may also leak after the dressing has been in place for a significant period of time. Often a leak may occur if an edge of the dressing becomes lifted or the tissue site is prone to movement. Edge lifting may be the lifting of a portion of dressing when the edge of the dressing is caught by clothing or other objects. Sometimes, the adhesive of the cover, such as the cover 110, may no longer re-bond to the epidermis 125 because of creasing of the cover 110 or surface contamination of the adhesive, for example with dead skin cells or other material. A drape strip can be used to patch the lifted, leaking edge of the cover 110. Historically, the drape strips are formed using an acrylic adhesive. However, some leaks may require an adhesive providing more sealing, some patients may be allergic to the acrylic adhesive, and some alternative adhesives may not provide an adequate bond to tissue, such as the epidermis 125. Many drape strips used to seal an edge of a cover are formed with a high-bond strength adhesive. A high-bond strength adhesive can provide assurance the drape strip will bond to both tissue and the adjacent cover. However, some tissue adjacent a tissue site may be friable or sensitive, and the use of a high-bond strength adhesive may damage the skin or otherwise injure the patient if the dressing and drape strip are removed. Some tissue sites may need a combination of drape strips having different adhesives. To adequately seal the cover, a clinician may keep a variety of drape strips, each having a different type and strength of adhesive, necessitating additional expense and storage issues.

As disclosed herein, the therapy system 100 can overcome these challenges and others by providing a drape strip, such as a sealing tape 132, having a selectable adhesive configuration. In some embodiments, the sealing tape 132 may have two adhesives, a first adhesive having a higher bond strength and a second adhesive having a relatively lower bond strength. As shown in FIG. 2, the sealing tape 132 may be applied to the cover 110 so that the sealing tape 132 may partially couple to the cover 110 and partially couple to an epidermis 125, covering an edge of the cover 110. The sealing tape 132 may provide increased sealing of the cover 110 to the epidermis 125.

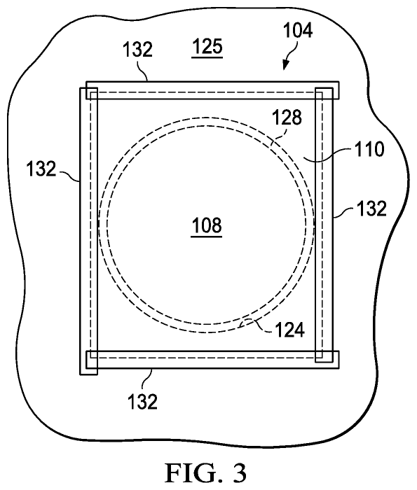

FIG. 3 is a plan view of the dressing 104 illustrating additional details that may be associated with some embodiments. As shown, the tissue interface 108 may be disposed at the tissue site 124 and covered with the cover 110 to form the sealed therapeutic environment 128. The sealing tape 132 may be positioned to cover edges of the cover 110. In some embodiments, the sealing tape 132 may be partially coupled to the cover 110 and partially coupled to the epidermis 125. In some embodiments, ends of the sealing tape 132 may overlap one another.

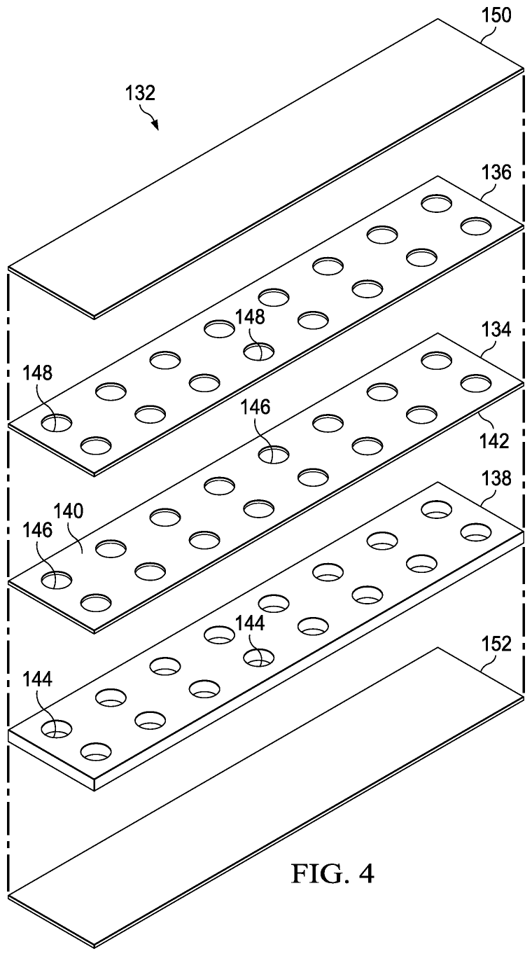

FIG. 4 is a perspective exploded view of the sealing tape 132, illustrating details that may be associated with some embodiments. In some embodiments, the sealing tape 132 may include a film layer 134, a first layer 136 formed from a bonding adhesive, and a second layer 138 formed from a sealing adhesive. The film layer 134 may be disposed between the first layer 136 and the second layer 138. For example, the film layer 134 may have a first side 140 and a second side 142. The first side 140 and the second side 142 may be surfaces of the film layer 134 facing opposite directions so that the first side 140 and the second side 142 form opposite sides of the film layer 134. The first layer 136 may be disposed on the first side 140. The second layer 138 may be disposed on the second side 142. In some embodiments, the sealing tape 132 can be manufactured from a “Silicone Tri-Lam” produced by Scapa® Healthcare model RX 1457S.

The film layer 134 may be liquid-impermeable and vapor-permeable, that is, the film layer 134 may allow vapor to egress and inhibit liquids from exiting. The film layer 134 may be a substrate formed from a flexible film that is breathable and may have a high moisture vapor transfer rate (MVTR), for example, greater than or equal to about 300 g/m²/24 hours. The film layer 134 may be formed from a range of medically approved films ranging in thickness from about 15 microns (μm) to about 50 microns (μm). Preferably, the film layer 134 may have a thickness of about 25.4 microns (μm) or 1 mil. In other embodiments, a drape having a low MVTR or that allows no vapor transfer might be used. In some embodiments, the film layer 134 can also function as a barrier to liquids and microorganisms.

The film layer 134 may be formed from numerous materials, such as one or more of the following: hydrophilic polyurethane (PU), cellulosics, hydrophilic polyamides, polyvinyl alcohol, polyvinyl pyrrolidone, hydrophilic acrylics, hydrophilic silicone elastomers, and copolymers of these. In some embodiments, the film layer 134 may be formed from a breathable cast matt polyurethane film sold by Expopack Advanced Coatings of Wrexham, United Kingdom, under the name INSPIRE 2301. The illustrative film may have an MVTR (inverted cup technique) of 14400 g/m²/24 hours and may be approximately 30 microns thick. Preferably, the film layer 134 may be formed of polyurethane and be substantially transparent.

The film layer 134 may be formed with a plurality of holes, for example, apertures 146. The apertures 146 may be numerous shapes, for example, circles, squares, stars, ovals, polygons, slits, complex curves, rectilinear shapes, triangles, or other shapes. Each aperture 146 of the plurality of apertures 146 may have an effective diameter. An effective diameter may be a diameter of a circular area having the same surface area as the aperture 146. The average effective diameter of each aperture 146 may be in the range of about 1 mm to about 50 mm. Preferably, each aperture 146 may have an average effective diameter of about 2 mm or greater. In other embodiments, the apertures 146 may be slot-shaped having a width of about 0.5 mm and a length of about 2 mm. The apertures 146 may have a uniform pattern or may be randomly distributed on the film layer 134. For example, in some embodiments, the apertures 146 may be distributed so that the apertures 146 extend to edges of the sealing tape 132. In other embodiments, the apertures 146 may be distributed so that a portion of the film layer 134 includes no apertures 146.

The first layer 136 may be coupled directly to the film layer 134. In some embodiments, the first layer 136 and the film layer 134 may be coextensive. In other embodiments, the first layer 136 and the film layer 134 may not be coextensive. The first layer 136 may be a bonding layer formed from a bonding adhesive, for example, a medically-acceptable, pressure-sensitive adhesive. For example, a bonding adhesive may be formed from an acrylic adhesive, rubber adhesive, high-tack silicone adhesive, polyurethane, or other substance. In some embodiments, a bonding adhesive may be formed from an acrylic adhesive with a coating weight of about 15 grams per square meter (“gsm”) to about 70 gsm. A bonding adhesive may also be a high-bond strength acrylic adhesive, patterrubber adhesive, high-tack silicone adhesive, or polyurethane, for example. In some embodiments, the bond strength or tackiness of a bonding adhesive may have a peel adhesion or resistance to being peeled from a stainless steel material between about 4 Newtons/25 millimeters (N/mm) to about 14N/25 mm on stainless steel substrate at 23° C. at 50% relative humidity based on the American Society for Testing and Materials (“ASTM”) standard ASTM D3330. Preferably, the first layer 136 can be an acrylic adhesive having a thickness of about 56 microns (μm) or about 2.2 mils.

The first layer 136 may be formed with a plurality of holes, for example, apertures 148. The apertures 148 may be numerous shapes, for example, circles, squares, stars, ovals, polygons, slits, complex curves, rectilinear shapes, triangles, or other shapes. Each aperture 148 of the plurality of apertures 148 may have an effective diameter in the range of about 1 mm to about 50 mm. Preferably, each aperture 148 may have an average effective diameter of about 2 mm or greater. In other embodiments, the apertures 148 may be slot-shaped having a width of about 0.5 mm and a length of about 2 mm. The apertures 148 may have a uniform pattern or may be randomly distributed on the first layer 136. For example, in some embodiments, the apertures 148 may be distributed so that the apertures 148 extend to edges of the sealing tape 132. In other embodiments, the apertures 148 may be distributed so that a portion of the first layer 136 includes no apertures 148.

The second layer 138 may be coupled directly to the film layer 134. In some embodiments, the second layer 138 and the film layer 134 may be coextensive. In other embodiments, the second layer 138 and the film layer 134 may not be coextensive. The second layer 138 may be a sealing layer formed from a sealing adhesive, for example, a soft material that provides a good seal with the tissue site 124 and/or the epidermis 125. A sealing adhesive may be formed of a silicone gel (or soft silicone), hydrocolloid, hydrogel, polyurethane gel, polyolefin gel, hydrogenated styrenic copolymer gels, or foamed gels with compositions as listed, or soft closed cell foams (polyurethanes, polyolefins) coated with an adhesive (e.g., 30 gsm-70 gsm acrylic), polyurethane, polyolefin, or hydrogenated styrenic copolymers. In some embodiments, a sealing adhesive may have a stiffness between about 5 Shore 00 and about 80 Shore 00. A sealing adhesive may be hydrophobic or hydrophilic. A sealing adhesive may be an adhesive having a low to medium tackiness, for example, a silicone polymer, polyurethane, or an additional acrylic adhesive. In some embodiments, the bond strength or tackiness of a sealing adhesive may have a peel adhesion or resistance to being peeled from a stainless steel material between about 0.1N/25 mm to about 2.6N/25 mm on stainless steel substrate at 23° C. at 50% relative humidity based on ASTM D3330. A sealing adhesive may achieve the bond strength above after a contact time of less than 60 seconds. Tackiness may be considered a bond strength of an adhesive after a very low contact time between the adhesive and a substrate. In some embodiments, a sealing adhesive may have a tackiness that may be about 30% to about 50% of the tackiness of a bonding adhesive. The second layer 138 has a thickness that may be in the range of about 100 microns (μm) to about 1000 microns (μm). Preferably, the second layer 138 may be a silicone adhesive having a thickness of about 255 microns (μm) or about 9.6 mils.

The second layer 138 may be formed with a plurality of holes, for example, apertures 144. The apertures 144 may be numerous shapes, for example, circles, squares, stars, ovals, polygons, slits, complex curves, rectilinear shapes, triangles, or other shapes. Each aperture 144 of the plurality of apertures 144 may have an effective diameter in the range of about 1 mm to about 50 mm. Preferably, each aperture 144 may have an average effective diameter of about 2 mm or greater. In other embodiments, the apertures 144 may be slot-shaped having a width of about 0.5 mm and a length of about 2 mm. The apertures 144 may have a uniform pattern or may be randomly distributed on the second layer 138. For example, in some embodiments, the apertures 144 may be distributed so that the apertures 144 extend to edges of the sealing tape 132. In other embodiments, the apertures 144 may be distributed so that a portion of the second layer 138 includes no apertures 144.

In some embodiments, the apertures 144, the apertures 146, and the apertures 148 may be arranged in two rows. The rows may be parallel to a length of the sealing tape 132. The apertures 144, the apertures 146, and the apertures 148 in a first row may be adjacent to the apertures 144, the apertures 146, and the apertures 148 in the second row so that centers of adjacent apertures are co-planar with a plane parallel to a width of the sealing tape 132. In other embodiments, centers of adjacent apertures are intentionally misaligned and are not co-planar with a plane parallel to a width of the sealing tape 132. In some embodiments, one or more of the apertures 144, the apertures 146, and the apertures 148 may be circular and others of the apertures 144, the apertures 146, and the apertures 148 may square, triangular, or slot-shaped. The apertures 144, the apertures 146, and the apertures 148 having different shapes may be aligned so that at least a portion of each aperture overlays an aperture in another layer.

The sealing tape 132 can also include a first release liner 150 and a second release liner 152. The first release liner 150 can be coupled to the first layer 136. For example, the first release liner 150 can be coupled to a side of the first layer 136 that is opposite the film layer 134. Preferably, the first release liner 150 may be releasably coupled to the first layer 136. In some embodiments, the first release liner 150 may be a protective layer. The first release liner 150 can be formed from a polyethylene terephthalate. In some embodiments, the first release liner 150 may have a thickness of about 2.0 mils or about 50 microns. The second release liner 152 can be coupled to the second layer 138. For example, the second release liner 152 can be coupled to a side of the second layer 138 that is opposite the film layer 134. Preferably, the second release liner 152 may be releasably coupled to the second layer 138. In some embodiments, the second release liner 152 may be a protective layer. The second release liner 152 can be formed from a polyurethane film and may have a thickness of about 3 mils or about 76.2 microns.

Each of the first release liner 150 and the second release liner 152 may have a coating to prevent permanent bonding to the adjacent adhesive. For example, a light coating of silicone adhesive can be applied to the first release liner 150 to prevent permanent bonding with the first layer 136. In some embodiments, the light coating of silicone adhesive applied to the first release liner 150 can be deposited in a pattern. Pattern deposition permits some portions of the first release liner 150 to have the coating, some portions to be free from the coating, and some portions to have an application of the coating that is thinner than in other areas. Application of the coating in this manner can increase the bond of the first release liner 150 to the first layer 136 while still permitting removal of the first release liner 150 due to a non-uniform bond created by the pattern deposition. Similarly, a light coating of an acrylic adhesive can be applied to the second release liner 152 to prevent permanent bonding with the second layer 138. In some embodiments, the light coating of acrylic adhesive applied to the second release liner 152 can be deposited in a pattern. Pattern deposition permits some portions of the second release liner 152 to have the coating, some portions to be free from the coating, and some portions to have an application of the coating that is thinner than in other areas. Application of the coating in this manner can increase the bond of the second release liner 152 to the second layer 138 while still permitting removal of the second release liner 152 due to a non-uniform bond created by the pattern deposition.

In some embodiments, the first release liner 150 and the second release liner 152 can include an indicator identifying the underlying adhesive. For example, the first release liner 150 can be printed with the terms bonding adhesive or acrylic adhesive, and the second release liner 152 can be printed with the terms sealing adhesive of silicone adhesive. In other embodiments, the first release liner 150 and the second release liner 152 can be clear. In other embodiments, the first release liner 150 and the second release liner 152 may be dyed with a color to provide an indication of the underlying adhesive. In some embodiments, each of the first release liner 150 and the second release liner 152 may include handling bars. Handling bars may be portions of the release liner proximate to an end of each release liner having an increased stiffness compared to the remainder of the release liner. The handling bar may be removable and may aid in handling and removal of the release liner. In some embodiments, the first release liner 150 and the second release liner 152 can have a plurality of punctures having no material removed. The punctures can increase the moisture vapor transmission rate without permitting the adjacent adhesives to flow through the puncture.

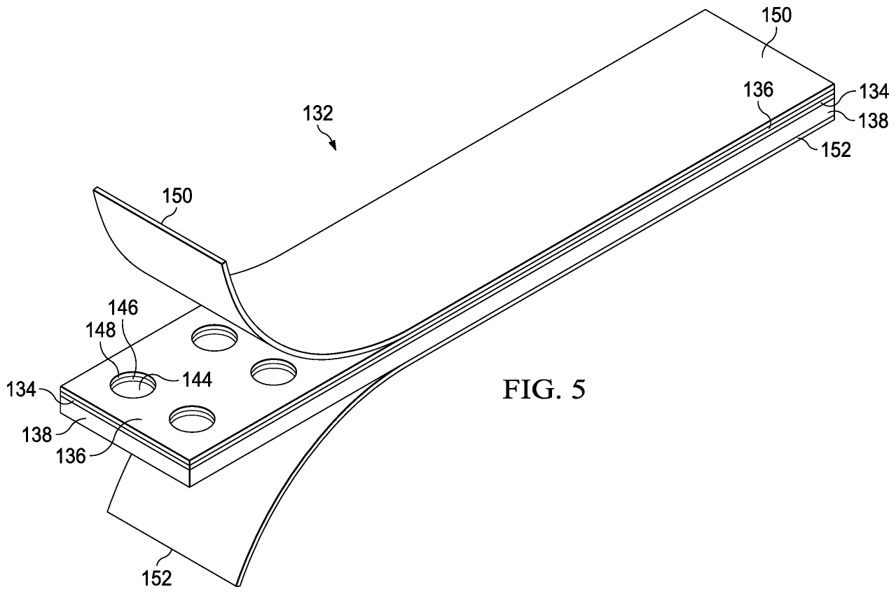

FIG. 5 is an assembled view of the sealing tape 132 illustrating additional details that may be associated with some embodiments. The first layer 136 and the second layer 138 may be coupled to opposite sides of the film layer 134. Preferably, the apertures 144, the apertures, 146, and the apertures 148 may be aligned so that fluid, for example, air, may flow across the sealing tape 132 through the apertures 144, the apertures 146, and the apertures 148. The first release liner 150 can be releasably coupled to the first layer 136, and the second release liner 152 can be releasably coupled to the second layer 138. The apertures 144, the apertures 146, and the apertures 148 can provide improved moisture vapor transmission over other sealing tapes. Because the apertures 144, the aperture 146, and the apertures 148 can provide a path for transmission of vapor across the sealing tape 132, materials permitting low or no moisture vapor transmission can be used in the manufacture of the sealing tape 132. For example, the sealing tape 132 may have a minimum moisture vapor transmission rate of 250 g/m²/day tested using the ASTM E96/E96M upright cup method modified at 38° C. at 10% relative humidity. In other embodiments, the apertures 144, the apertures 146, and the apertures 148 can be increased in number and/or increased in size to increase the minimum moisture vapor transmission rate. The apertures 144, the apertures 146, and the apertures 148 can also be decreased in size and/or decreased number to decrease the minimum moisture vapor transmission rate. In some embodiments, the apertures 144, the apertures 146, and the apertures 148 permit at least 20% of the surface area of the sealing tape 132 to be open and free of adhesive, and the remaining 80% of the surface are of the sealing tape 132 to be covered by an adhesive.

The sealing tape 132 can be manufactured by first providing the film layer 134. The first layer 136 can be coupled to the first side 140 of the film layer 134. For example, the first layer 136 can be deposited, laminated, bonded, cast and cured, or otherwise secured to the film layer 134. The second layer 138 can be coupled to the second side 142 of the film layer 134. For example, the second layer 138 can be deposited, laminated, bonded, cast and cured, or otherwise secured to the film layer 134. The apertures 148, the apertures 146, and the apertures 144 can be formed in the assembly having the first layer 136, the film layer 134, and the second layer 138. For example, the assembly can be punched, punctured, cut, pressed or otherwise manipulated to form each of the apertures 144, the apertures 146, and the apertures 148 in a single process. The first release liner 150 can be coupled to the first layer 136, and the second release liner 152 can be coupled to the second layer 138. In some embodiments, the apertures 144, the apertures 146, and the apertures 148 are formed simultaneously following assembly of the first layer 136, the film layer 134, and the second layer 138. In other embodiments, the apertures 144, the apertures 146, and the apertures 148 can be formed prior to assembly of the film layer 134, the first layer 136, and the second layer 138. The apertures 144, the apertures 146, and the apertures 148 can be aligned so that the apertures 144, the apertures 146, and the apertures 148 are coincident when assembled. The apertures 144, the apertures 146, and the apertures 148 can also be intentionally misaligned during assembly to control the rate of moisture vapor transfer across the sealing tape 132. In still another embodiment, the first layer 136 can be coupled to a first film layer, and the second layer 138 can be coupled to a second film layer. The apertures 148, the apertures 146, and the apertures 144 can be formed in the first layer 136, the second layer 138 and their associated first film layer and second film layer, respectively. The first film layer and the second film layer can then be coupled to each other so that the apertures in the first film layer are substantially aligned with the apertures in the second film layer to form the film layer 134 having the apertures 144.

In operation, the dressing 104 can be positioned at the tissue site 124. For example, the tissue interface 108 can be disposed in the tissue site 124, and the cover 110 can be placed over the tissue interface 108 and the tissue site 124 and sealed to the epidermis 125 surrounding the tissue site 124. The dressing 104 can be evaluated for leaks and where a leak is identified, the sealing tape 132 can be applied. If the sealing tape 132 is applied, the sealing properties of the tape 132 are selectable at time of application by choosing which release liner to remove, the first release liner 150 or the second release liner 152. Depending on the type of leak, the cover 110, the location of the tissue site 124, the friability of the periwound skin at the tissue site 124, the patient intolerance to pain during removal of the dressing 104, the care-giver preferences for later removal of the sealing taper 132, and the duration the dressing 104 has been disposed at the tissue site 124, the sealing tape 132 can be applied with either the first layer 136 or the second layer 138. The sealing tape 132 adhesion chosen for the application the dressing 104 may be different around the edge of the dressing 104. For example, if the dressing 104 is disposed at a moisture-prone area, the dressing 104 can be applied with the first layer 136 of the sealing tape 132. In some embodiments, more than one strip of the sealing tape 132 may be used. For example, a first strip of the sealing tape 132 can be applied with the second layer 138, improving sealing of the sealed therapeutic environment at the edge of the dressing 104. The first strip of the sealing tape 132 can be partially overlapped with a second strip of the sealing tape 132 applied with the first layer 136, creating a stronger bond along the edge of the dressing 104. In other exemplary applications, the first release liner 150 can be removed from the sealing tape 132, and the first layer 136 can be placed in contact with the cover 110 and the epidermis 125. The second release liner 152 can become the outer surface of the sealing tape 132, forming a protective layer for the underlying second layer 138. In some embodiments, the second layer 138 can flow through the apertures 146 and the apertures 148, providing additional sealing effects. Alternatively, the second release liner 152 can be removed from the sealing tape 132, and the second layer 138 can be placed in contact with the cover 110 and the epidermis 125 surrounding the tissue site. The first release liner 150 can become the outer surface of the sealing tape 132, forming a protective layer for the underlying first layer 136.

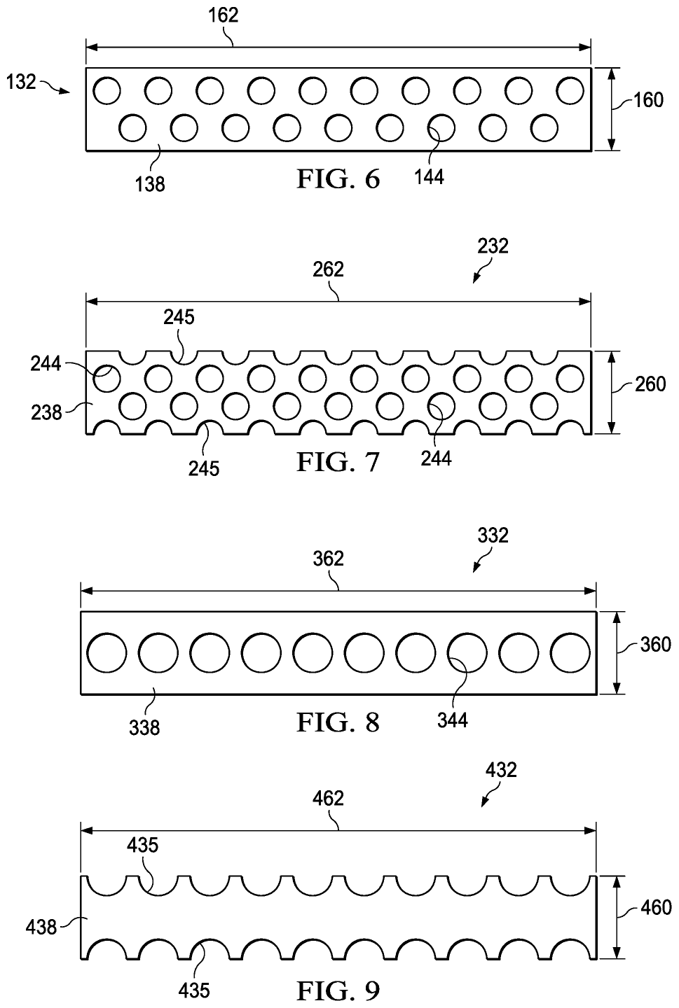

FIG. 6 is a plan view of the sealing tape 132 illustrating additional details that may be associated with some embodiments. As illustrated, the first release liner 150 and the second release liner 152 are not shown. The second layer 138 of the sealing tape 132 is visible, and each of the apertures 144, the apertures 146, and the apertures 148 are aligned. The sealing tape 132 may have a width 160 and a length 162. In some embodiments, the width 160 may be between about 20 mm. In other embodiments, the width 160 may be between about 10 mm to about 30 mm. In some embodiments, the length 162 may be up to about 200 mm. The sealing tape 132 may be cut or torn so that a sealing tape 132 having the length 162 suitable for use may be provided. For example, a sealing tape 132 may be provided in rolls, allowing a clinician to select a length suitable for a particular tissue site and cut or tear an end of the sealing tape 132 to remove it from the roll. In some embodiments, the apertures 144 may have a diameter between about 0.5 mm to about 10 mm. In other embodiments, the apertures 144 may have a diameter between about 5 mm to about 30 mm. The sealing tape 132 may include two rows of apertures 144 that may be offset from one another. In some embodiments, the apertures 144 may be evenly distributed on the sealing tape 132.

FIG. 7 is a plan view of a sealing tape 232 illustrating additional details that may be associated with some embodiments. The sealing tape 232 may be similar to and operate in a manner similar to the sealing tape 132 of FIG. 9. Similar elements have been indexed to 2XX. For example, the sealing tape 232 may have a width 260, a length 262, a first layer 236 having apertures 248, a film layer 234 having apertures 246, and a second layer 238 having apertures 244. As illustrated, a first release liner 250 and a second release liner 252 are not shown. The apertures 244 may be positioned similar to the apertures 144. In some embodiments, the sealing adhesive 230 of the sealing tape 232 may include edge apertures 245. If the apertures 244 are circles, as shown, the edge apertures 245 may be semi-circles having a radius equal to a radius of the apertures 244. The edge apertures 245 may be positioned so that a diameter of each edge aperture 245 is adjacent to an edge of the sealing tape 232 that is parallel to the length 262. In other embodiments, the edge apertures 245 may be positioned so that a chord of each edge aperture 245 is adjacent to an edge of the sealing tape 232 that is parallel to the length 262. The film layer 234 and the first layer 236 may have edge apertures 247 and edge apertures 249 that are coextensive with the edge apertures 245. In some embodiments, the edge apertures 245 may be disposed along both edges. In other embodiments, the edge apertures 245 may only be disposed along one edge. The apertures 244 and the edge apertures 245 may be evenly distributed on the sealing tape 232. For example, each aperture 244 and edge aperture 245 may be separated from adjacent apertures 244 and edge apertures 245 by a same distance. In some embodiments, an even distribution may produce a sealing tape 232 having apertures 244 and edge apertures 245 extending between edges of the sealing tape 232 at regularly repeating distances.

FIG. 8 is a plan view of a sealing tape 332 illustrating additional details that may be associated with some embodiments. The sealing tape 332 may be similar to and operate in a manner similar to the sealing tape 132 of FIG. 6. Similar elements have been indexed to 3XX. For example, the sealing tape 332 may have a width 360, a length 362, a first layer 336 having apertures 348, a film layer 334 having apertures 346, and a second layer 338 having apertures 344. As illustrated, a first release liner 350 and a second release liner 352 are not shown. In some embodiments, the apertures 344 may have a diameter between about 10 mm and about 15 mm. In other embodiments, the apertures 344 may have a diameter between about 5 mm and about 30 mm. In some embodiments, the sealing tape 332 may include a single row of apertures 344. In some embodiments, the apertures are disposed near a center of the width 360 of the sealing tape 332 and may be evenly distributed parallel to the length 362 of the sealing tape 332.

FIG. 9 is a plan view of the sealing tape 432 illustrating additional details that may be associated with some embodiments. The sealing tape 432 may be similar to and operate in a manner similar to the sealing tape 232 of FIG. 7. Similar elements have been indexed to 4XX. For example, the sealing tape 432 may have a width 460, a length 462, a first layer 436 having edge apertures 449, a film layer 434 having edge apertures 447, and a second layer 438 having edge apertures 445. As illustrated, a first release liner 450 and a second release liner 452 are not shown. The edge apertures 445 may be positioned similar to the edge apertures 245 and have a radius equal to a radius of the apertures 344.

In some embodiments, the adhesives may be mixed with blowing or expanding agents, for example organic and inorganic low temperature boiling point liquids. The blowing or expanding agents allow for the adhesives to expand under the application of heat or light to increase the thickness of the adhesive following deposition by one of the above described processes. The blowing or expanding agents may reduce the amount of adhesive needed and decrease the cost of production and the cost of the resulting sealing tape 132. In some embodiments, the application of heat or light may be delayed until application of the sealing tape 132 to the epidermis 125 so that the contact area with the patient's epidermis 125 may increase as the first layer 136 and the second layer 138 warm by contact with the patient's epidermis 125. The application of light or heat following application of the sealing tape 132 to the epidermis 125 can provide a better seal for some embodiments of the sealing tape 132 to the epidermis 125.

The systems, apparatuses, and methods described herein may provide significant advantages. For example, the drape strips can provide a clinician with a choice of bond strength when providing supplemental sealing to a dressing. The drape strips provide a high-seal, low-tack, low trauma adhesive and a high-tack adhesive in the same application, removing the need for a user to have to have strips of acrylic adhesive drape and strips of silicone or other lo-tack adhesive strips available. Some users who are sensitive to different adhesives may be treated with the same product, reducing costs. The flowable nature of the sealing adhesive make the structure a good sealing material even when used with the bonding adhesive side adjacent to the tissue. The drape strip may have benefits of scar reduction while maintaining a relatively high MVTR.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 104, the container 106, or both may be eliminated or separated from other components for manufacture or sale. In other example configurations, the controller 112 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site with negative pressure, the dressing comprising:
a tissue interface configured to be positioned adjacent to the tissue site;
a sealing member configured to be positioned over the tissue interface and the tissue site to form a sealed environment; and
a sealing tape comprising:
a carrier layer having a first side and a second side;
a silicone adhesive layer bonded to the first side of the carrier layer;
an acrylic adhesive layer bonded to the second side of the carrier layer;
a plurality of holes extending through the silicone adhesive layer, the acrylic adhesive layer, and the carrier layer;
a first release liner coupled to silicone adhesive layer; and
a second release liner coupled to the acrylic adhesive layer.

2. The dressing of claim 1, wherein the carrier layer comprises polyurethane.

3. The dressing of claim 2, wherein the carrier layer has a thickness of about 1 mil.

4. The dressing of claim 1, wherein the carrier layer has a thickness of about 1 mil.

5. The dressing of claim 1, wherein the silicone adhesive layer has at thickness of about 9.6 mils.

6. The dressing of claim 1, wherein the acrylic adhesive layer has at thickness of about 2.2 mils.

7. The dressing of claim 1, wherein each hole of the plurality of holes has an average effective diameter of about 2 millimeters (“mm”).

8. The dressing of claim 7, wherein the plurality of holes comprises a first row of holes and a second row of holes, the first row and the second row being adjacent to each other and equidistantly spaced across a width and length of the sealing tape.

9. The dressing of claim 1, wherein the sealing tape has a minimum moisture vapor transmission rate (“MVTR”) of about 250 g/m²/day.

10. The dressing of claim 1, wherein the first release liner has an acrylic adhesive coating on a side adjacent to the silicone adhesive layer.

11. The dressing of claim 1, wherein the second release liner has a silicone adhesive coating on a side adjacent to the acrylic adhesive layer.

12. The dressing of claim 1, further comprising:
a first pair of handling bars coupled to the first release liner; and
a second pair of handling bars coupled to the second release liner.

13. A drape strip comprising:
a substrate having a first side and a second side;
a sealing layer bonded to the first side of the substrate;
a bonding layer bonded to the second side of the substrate;
a plurality of apertures extending through the sealing layer, the bonding layer, and the substrate;
a first protective layer coupled to sealing layer; and
a second protective layer coupled to the bonding layer.

14. The drape strip of claim 13, wherein the substrate comprises polyurethane.

15. The drape strip of claim 14, wherein the substrate has a thickness of about 1 mil.

16. The drape strip of claim 13, wherein the substrate has a thickness of about 1 mil.

17. The drape strip of claim 13, wherein the sealing layer has at thickness of about 9.6 mils.

18. The drape strip of claim 13, wherein the bonding layer has at thickness of about 2.2 mils.

19. The drape strip of claim 13, wherein each hole of the plurality of apertures has an average effective diameter of about 2 millimeters (“mm”).

20. The drape strip of claim 19, wherein the plurality of apertures comprises a first row of apertures and a second row of apertures, the first row and the second row being adjacent to each other and equidistantly spaced across a width and length of the drape strip.

21. The drape strip of claim 13, wherein the drape strip has a minimum moisture vapor transmission rate (“MVTR”) of about 250 g/m²/day.

22. The drape strip of claim 13, wherein the first protective layer has an acrylic adhesive coating on a side adjacent to the sealing layer.

23. The drape strip of claim 13, wherein the second protective layer has a silicone adhesive coating on a side adjacent to the bonding layer.

24. The drape strip of claim 13, further comprising:
a first pair of gripping supports coupled to the first protective layer; and
a second pair of gripping supports coupled to the second protective layer.

25. A method of manufacturing a sealing tape comprising:
providing a carrier layer having a first side and a second side;
bonding a silicone adhesive layer to the first side of the carrier layer;
bonding an acrylic adhesive layer to the second side of the carrier layer;
forming a plurality of holes extending through the silicone adhesive layer, the acrylic adhesive layer, and the carrier layer;
releasably coupling a first release liner to silicone adhesive layer; and
releasably coupling a second release liner to the acrylic adhesive layer.

26. The method of claim 25, wherein the method further comprises: providing an indicator on the first release liner and the second release liner associated with the silicone adhesive layer and the acrylic adhesive layer, respectively.

27. The method of claim 26, wherein providing an indicator comprises printing a label.

28. The method of claim 25, wherein forming the plurality of holes comprises forming a first row of holes and a second row of holes, the first row and the second row being adjacent to each other and equidistantly spaced across a width and length of the sealing tape.

29. The method of claim 25, wherein the method further comprises coating the first release liner with an acrylic adhesive on a side adjacent to the silicone adhesive layer.

30. The method of claim 25, wherein the method further comprises coating the second release liner with a silicone adhesive on a side adjacent to the acrylic adhesive layer.

31. The method of claim 25, further comprising:
coupling a first pair of handling bars to the first release liner; and
coupling a second pair of handling bars to the second release liner.

32. (canceled)
